(19) United States
(12) Patent Application Publication
(10) Pub. No.: US 2012/0204896 A1
(43) Pub. Date: Aug. 16, 2012

(54) POLYAMIDES OF FATTY ACID DIMERS AND DIAMINES FOR FIXING A HAIRSTYLE

(76) Inventors: Matthias Schweinsberg, Hamburg (DE); Luca Marchese, Arese (IT); Carine Dogan, Vigneux sur Seine (FR); Rolf Bayersdoerfer, Hamburg (DE)

(21) Appl. No.: 13/450,816

(22) Filed: Apr. 19, 2012

Related U.S. Application Data
(63) Continuation of application No. PCT/EP2010/064543, filed on Sep. 30, 2010.

(30) Foreign Application Priority Data
Oct. 20, 2009 (DE) 10 2009 045 842.5

(51) Int. Cl. A61K 8/88; A61Q 5/06

(52) U.S. Cl. 132/203; 424/70.17; 424/43

(57) ABSTRACT

Use of a cosmetic product for fixing a hairstyle containing at least one polyamide in a cosmetic carrier, wherein the polyamide is a reaction product of at least one dimerized fatty acid and at least one diamino compound. The invention also relates to a corresponding hair treatment method, and to cosmetic products containing at least one reaction product of at least one dimerized fatty acid and at least one diamino compound, and at least one foaming agent.

### CROSS REFERENCE TO RELATED APPLICATIONS

The present application is a continuation of International Application No. PCT/EP2010/064543 filed 30 Sep. 2010, which claims priority to German Patent Application No. 10 2009 045 842.5 filed 20 Oct. 2009, both of which are incorporated herein by reference.

The present invention relates to the use of a cosmetic agent, comprising in a cosmetic carrier at least one polyamide that is a reaction product of at least one dimerized fatty acid and at least one diamino compound, for fixing the shape of a hairstyle, as well as a corresponding hair treatment method. Furthermore, the invention relates to cosmetic agent that is at least one reaction product of at least one dimerized fatty acid and at least one diamino compound, and comprises at least one propellant.

Today, a suitably looking hairstyle is generally regarded as an essential part of a well groomed appearance. Based on current fashion trends, time and again hairstyles are considered chic which, for many types of hair, can only be formed or sustained over a longer period of up to several days by use of certain setting materials. Thus, hair treatment agents which provide a permanent or temporary hairstyling play an important role. Temporary styling intended to provide a good hold, without compromising the healthy appearance of the hair such as the gloss can be obtained, for example, by use of hairsprays, hair waxes, hair gels, hair foams, setting lotions, etc.

Suitable compositions for temporary hairstyling usually contain synthetic polymers as the styling component. Preparations comprising a dissolved or dispersed polymer can be applied on the hair by propellants or by a pumping mechanism. Hair gels and hair waxes are, however, not generally applied directly on the hair, but rather dispersed with a comb or by hand.

An important property of an agent for temporary styling of keratin fibers, hereafter also called styling agents, consists in giving the treated fibers the strongest possible hold in the created shape. If the keratinic fibers concern human hair, then one also speaks of a strong hairstyle hold or a high degree of hold of the styling agent. Styling hold is determined basically by the type and quantity of synthetic polymer used; however, there may also be an influence from other components of the styling agent.

In addition to a high degree of hold, styling agents must fulfill a whole series of additional requirements. These requirements can be broadly subdivided into properties on the hair, properties of the formulation in question (e.g., properties of the foam, gel or sprayed aerosol), and properties regarding the handling of the styling agent, wherein particular importance is attached to the properties on the hair. These include moisture resistance, low stickiness and a balanced conditioning effect. Furthermore, a styling agent should be universally applicable for as many types of hair as possible.

To do justice to the various requirements, various synthetic polymers have been developed and are being used in styling agents. These polymers can be subdivided into cationic, anionic, non-ionic and amphoteric film-forming and/or setting polymers. Ideally these polymers form a polymer film when applied even in low amounts to hair, imparting a strong hold to the hairstyle while also being sufficiently flexible so not to break under stress. If the polymer film is too brittle, film plaques develop (i.e., residues that are shed with movement of the hair and give the impression that the user of the respective styling agent has dandruff).

Further, the temporarily styled hair should look healthy and natural in addition to the strong hold. Here, hair gloss plays a prominent role. Consequently, sufficient amounts of brighteners are often added to the hairstyling agents. These brighteners include oils or shine-enhancing pigments such as mica particles. Shine-enhancing particles have the disadvantage that over time they become detached from the hair and after a while are found, for example, on the clothes or skin. Oils are a burden on the hair and in part lead to a worsened adhesion of the film-forming or setting polymers on the hair. This can possibly lead to the disadvantage that the constructed hairstyle cannot be fixed for a sufficient length of time by the film-forming or setting polymers (i.e., the hairstyle falls out more quickly).

Accordingly, the present invention provides an agent for the temporary styling of and/or care of keratinic fibers that gives a high degree of hold, possesses good elasticity and does not exhibit the abovementioned disadvantages.

It has now been surprisingly found that this can be achieved by use of a specific polyamide.

A first subject matter of the present invention is the use of a cosmetic agent for fixing the shape of a hairstyle, comprising in a cosmetic carrier at least one polyamide that is a reaction product of at least one dimerized fatty acid and at least one diamino compound.

In the context of the invention, all quantitative data are understood to always take into account each of the cited upper and lower limits.

In all Formulae below, the symbol * signifies a chemical bond that stands for a free valence of a structural fragment.

Dimerized fatty acids are obtained as a product in an oligomerization or polymerization reaction of unsaturated long chain, monobasic fatty acids.

Dimerized fatty acids are well known to the person skilled in the art and are commercially available. When manufactured, dimerized fatty acids are known to exist as a mixture of a plurality of isomers and oligomers. Before work up, this mixture comprises 0 to 15 wt % monomeric fatty acids, 60 to 96 wt % dimerized fatty acids and 0.2 to 35 wt % trimerized fatty acids or higher oligomerized fatty acids. The crude mixture is normally worked up by distillation, sometimes followed by hydrogenation (saturation of the remaining double bonds with hydrogen).

In the context of the inventive use, the cosmetic agent preferably contains the polyamide in an amount of 0.01 to 30 wt %, preferably 0.1 to 15.0 wt %, more preferably 0.5 to 10.0 wt %, and quite particularly preferably 1.0 to 5.0 wt %, based on weight of the agent. These quantity ranges also apply for the following preferred embodiments of the polyamide.

Polyamides according to the invention are present in the cosmetic agent in a molecular weight distribution. Preferred polyamides have an average molecular weight (weight average) of 10 kDa to 1000 kDa, particularly 50 kDa to 800 kDa, quite particularly preferably 100 kDa to 400 kDa. The stated weight average is an average molecular weight that takes into account the total weight of the molecules of various molecular weight and not simply the number of molecules. The statistical calculation of the weight average from the molecular weight distribution is well known and can be found in text books.

It is inventively preferable to use such cosmetic agents wherein the polyamide has a glass transition temperature of −60° C. to 90° C., particularly −40° C. to 15° C.

Moreover, a particularly good effect is apparent if cosmetic agents are used wherein the polyamide has an E-modulus at 2% deformation of 10 to 500, in particular 20 to 150. E-modulus is measured according to ASTM D638.

Particularly preferred useable polyamides have an elongation at break in % of 20 to 1000, particularly 400 to 1000, quite particularly 600 to 1000. Elongation at break is measured according to DIN 53455.

Suitable dimerized fatty acids can be obtained by coupling or condensation of two moles of unsaturated monocarboxylic acids (a mixture of various unsaturated monocarboxylic acids can also be employed as the suitable monocarboxylic acid). Unsaturated fatty acids can be provided with the aid of diverse known catalytic or non-catalytic polymerization processes. Production processes for dimerized fatty acids are known, for example, from U.S. Pat. Nos. 2,793,219 and 2,955,219.

Those dimerized fatty acids have been found to be preferred which are produced by coupling unsaturated (C₁₀ to C₂₄) monocarboxylic acids. They are mono-unsaturated (C₁₀ to C₂₄) monocarboxylic acids and/or polyunsaturated (C₁₀ to C₂₄) monocarboxylic acids.

Dimerized fatty acids containing 36 carbon atoms, obtained by dimerizing an unsaturated monocarboxylic acid containing 18 carbon atoms, such as oleic acid, linoleic acid, linolenic acid and their mixtures (mixture of for example tallow oil fatty acid cut), are particularly preferably utilized for manufacturing the inventively used polyamides.

Such dimerized fatty acids contain a C₃₆ dicarboxylic acid as the major constituent and usually have an acid number of 180 to 215, a saponification number of 190 to 205 and a neutral equivalent of 265 to 310. Dimerized fatty acids with less than 30 wt % of by-products including monocarboxylic acids, trimerized fatty acids as well as higher oligomerized/polymerized fatty acids are particularly suitable in the context of the invention. The dimerized fatty acids can be hydrogenated and/or distilled before being reacted to form the inventively used polyamides. According to the invention, the dimerized fatty acid used for production of the polyamide preferably has a content of at least 90 wt % of the dimer.

Particularly preferred dimerized fatty acids used for the production of the polyamide are manufactured by coupling linoleic acid and/or linolenic acid and/or oleic acid. Mixtures of oleic acid and linoleic acid are found in the tallow oil fatty acid cut, which represents a cost-effective raw material source. A typical composition of dimerized fatty acids formed by treating the tallow oil fatty acids having 18 carbon atoms and which are suitable for manufacturing the inventively used polyamides is:

| | |
|---|---|
| C₁₈ monocarboxylic acids (monomer) | 0-15% wt % |
| C₃₆ dimerized fatty acid (dimer) | 60-96% wt % |
| C₅₄ (or higher) trimerized or higher oligomerized fatty | 0.2-35% wt % |
| acids | |

Furthermore, in one embodiment of the invention it can be preferred to add, in addition to the dimerized fatty acid, at least one aliphatic dicarboxylic acid containing 6 to 18 carbon atoms for manufacturing the polyamide. Here, both linear as well as branched dicarboxylic acids can be used. Exemplary suitable dicarboxylic acids have the Formula HOOC—R^{a}—COON wherein R^{a} is a divalent, aliphatic, hydrocarbon structural fragment with 4 to 16 carbon atoms, such as azelaic acid, sebacic acid, dodecane-1,12-dicarboxylic acid and their mixtures. R^{a} can be linear or branched.

The dimerized fatty acid (and the optionally additionally added aliphatic dicarboxylic acid with 6 to 18 carbon atoms) used for manufacturing the polyamides is preferably treated with at least one diamino compound. Those polyamides manufactured with at least one diamino compound chosen from diamino compounds of Formula (I) exhibited better properties for the inventive use

H₂N—R¹—NH₂ (I)

wherein R¹ is a linear (C₂ to C₁₀) alkylene group, a branched (C₂ to C₁₀) alkylene group, a *—R²—O—(CH₂CH₂O)ₙ(CH₂CHMeO)ₘ—R³—* group wherein R² and R³ independently of one another is a (C₂ to CO alkylene group (particularly ethane-1,2-diyl or propane-1,2-diyl), and n and m independently of one another is an integer from 0 to 100, wherein the sum of m+n>0, or a group of Formula

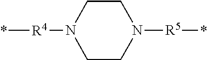

wherein R⁴ and R⁵ independently of one another is a (C₂ to C₆) alkylene group.

In the *—R²—O—(CH₂CH₂O)ₙ(CH₂CHMeO)ₘ—R³—* group, the ethylene oxide or propylene oxide groups can be present as a block or distributed statistically.

Polyamides formed by the reaction of at least one dimerized fatty acid with a combination chosen from at least one compound of Formula (I) and at least one compound of Formula (I-1) exhibit excellent performance properties

H₂N—R¹—NH₂ (I)

H₂N—R²—O—(CH₂CH₂O)ₙ(CH₂CHMeO)ₘ—R³—NH₂ (I-1)

wherein R¹ is a (C₂ to C₁₀) alkylene group, R² and R³ independently of one another is a (C₂ to C₁₀) alkylene group, and n and m independently of one another is an integer from 0 to 100, wherein the sum of m+n>0.

Compounds of Formula (I-1) represent polyoxyalkylenediamines. Processes for preparation of these polyoxyalkylenediamines are known to one skilled in the art and include the reaction of initiator molecules containing two hydroxyl groups with ethylene oxide and/or monosubstituted ethylene oxide (e.g., propylene oxide) followed by conversion of the terminal hydroxyl group into amino groups.

If compounds of Formula (I-1) having m>0 are used, then it is again preferred to choose those compounds of Formula (I-1) wherein additionally n>0, with the proviso that the total diamino compound of Formula (I) has a maximum fraction of 50 wt % of propylene oxide units, relative to the weight of the diamino compound. The ethylene oxide and propylene oxide units according to Formula (I-1) or according to Formula (I) can be distributed statistically, sequentially or be in at least two blocks.

If R¹ of the compound according to Formula (I) is a *—R²—O—(CH₂CH₂O)ₙ(CH₂CHMeO)ₘ—R³—* group, then the maximum fraction of propylene oxide units is preferably 40 wt % and particularly preferably maximum 30 wt %, based on the weight of the compound according to Formula (I).

Inventively preferred suitable polyoxyalkylenediamines of Formula (I-1) have a molecular weight of 460 to 6000 g/mol, particularly preferably 600 to 5000.

Inventively preferred suitable polyoxyalkylenediamines are marketed as the commercial product Jeffamine® by Huntsman Corporation, Houston, Tex. These polyoxyalkylenediamines are manufactured by treating bifunctional initiators with ethylene oxide and propylene oxide and subsequently converting the terminal hydroxyl groups into amino groups. Particularly preferred polyoxyalkyleneamines are part of the Jeffamine™ D series and JD series, (particularly Jeffamine J02000, Jeffamine JD 400 and Jeffamine JD230) from Huntsman Chemical Company.

Exemplary preferred linear alkylenediamines (R¹ in Formula (I) is a linear C₂-C₁₀ alkylene group) are 1,2-ethylenediamine, 1,2-propylenediamine, 1,3-propylenediamine, tetramethylenediamine, pentamethylenediamine, hexamethylenediamine, octamethylenediamine.

Exemplary preferred branched alkylenediamines (R¹ in Formula (I) is a branched C₂-C₁₀ alkylene group) are 2-methyl-1,5-pentanediamine, 5-methyl-1,9-nonanediamines and 2,2,4-trimethyl-1,6-hexanediamine and mixtures thereof.

Furthermore, it was found to be particularly advantageous when at least one diamino compound is 1,2-ethylenediamine.

The stated polyamides can be obtained by standard processes under known reaction conditions. The dimerized fatty acid and diamino compound(s) are usually caused to react at temperatures of 100° C. to 300° C. for a period of 1 to 8 hours. The reaction is mainly carried out at 140° C. to 240° C. until the theoretical amount of water from the condensation reaction forms. The reaction is preferably carried out under an inert atmosphere such as nitrogen. In order to complete the reaction, the reaction system is preferably placed under vacuum so as to facilitate the removal of water and other volatile constituents. Use of acid catalysts (such as phosphoric acid) and a vacuum (the latter particularly for the final reaction phase) is preferred in order to ensure an almost complete conversion to the amide.

The number of free carboxyl groups or free amine groups in the polyamide is a function of the relative amounts of the carboxylic acid components and diamine components employed in the production of the polyamide. The inventively employed polyamide can be acid-terminated, amine-terminated or acid- and amine-terminated. Mixtures of these correspondingly terminated polyamides can also be used.

Inventively useable acid-terminated polyamides preferably have Formula (IIa),

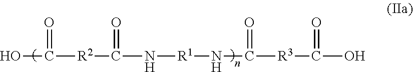

wherein
R¹ is a linear (C₂ to C₁₀) alkylene group, a branched (C₂ to C₁₀) alkylene group, a *—R²—O—(CH₂CH₂O)ₙ(CH₂CHMeO)ₘ—R³—* group wherein R² and R³ independently of one another is a (C₂ to C₁₀) alkylene group (particularly ethane-1,2-diyl or propane-1,2-diyl), and n and m independently of one another is an integer from 0 to 100, wherein the sum of m+n>0, or a group of Formula

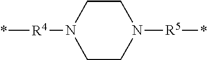

wherein R⁴ and R⁵ independently of one another is a (C₂ to C₆) alkylene group,
R² is independently for each repeat unit a (C₂₀ to C₄₀) alkylene group,
R³ is a (C₂₀ to C₄₀) alkylene group, and
n is the number of repeat units and is an integer from 10 to 100,000.

Inventively useable amine-terminated polyamides quite particularly preferably have Formula (IIb),

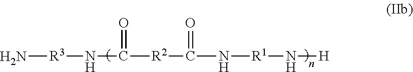

wherein
R¹ is a linear (C₂ to C₁₀) alkylene group, a branched (C₂ to C₁₀) alkylene group, a *—R²—O—(CH₂CH₂O)ₙ(CH₂CHMeO)ₘ—R³—* group wherein R² and R³ independently of one another is a (C₂ to C₁₀) alkylene group (particularly ethane-1,2-diyl or propane-1,2-diyl), and n and m independently of one another is an integer from 0 to 100, wherein the sum of m+n>0, or a group of Formula

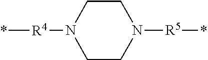

wherein R⁴ and R⁵ independently of one another is a (C₂ to C₆) alkylene group,
R² is independently for each repeat unit a (C₂₀ to C₄₀) alkylene group,
R³ is a linear (C₂ to C₁₀) alkylene group, a branched (C₂ to C₁₀) alkylene group, a *—R⁴—O—(CH₂CH₂O)ₚ(CH₂CHMeO)ₘ—R⁵—* group wherein R⁴ and R⁵ independently of one another is a (C₂ to C₁₀) alkylene group (particularly ethane-1,2-diyl or propane-1,2-diyl), and p and m independently of one another is an integer number from 0 to 100, wherein the sum of m+p>0, and
n is the number of repeat units and is an integer from 10 to 100,000.

Furthermore, the amine-terminated polyamides can also be present as ammonio-terminated polyamides. In this case the terminal amino groups are quaternized with (C₁ to C₂₀) alkyl groups.

Inventively useable amine- and acid-terminated polyamides preferably have Formula (IIc),

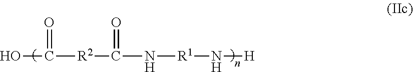

wherein
R¹ is a linear (C₂ to C₁₀) alkylene group, a branched (C₂ to C₁₀) alkylene group, a *—R²—O—(CH₂CH₂O)ₙ(CH₂CHMeO)ₘ—R³—* group wherein R² and R³ independently of one another is a (C₂ to C₁₀) alkylene group (particularly ethane-1,2-diyl or propane-1,2-diyl), and n and m independently of one another is an integer number from 0 to 100, wherein the sum of m+n>0, or a group of Formula

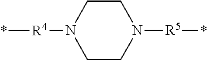

wherein R⁴ and R⁵ independently of one another is a (C₂ to C₆) alkylene group,
R² is independently for each repeat unit a (C₂₀ to C₄₀) alkylene group, and
n is the number of repeat units and is an integer from 10 to 100,000.

Preferred inventively useable polyamides have an acid number of 0.01 to 5, particularly 0.05 to 4. Acid number is determined by measurement methods according to DIN EN ISO 2114.

In addition, preferred useable polyamides have an amine number from 0.1 to 90, particularly 2 to 20. Amine number is determined by measurement methods according to DIN 53176.

Agents according to the invention comprise the ingredients or active substances in a cosmetically acceptable carrier.

Preferred cosmetically acceptable carriers are aqueous, alcoholic or aqueous alcoholic media (containing preferably at least 10 wt % water, based on total agent). In particular, lower alcohols containing 1 to 4 carbon atoms such as ethanol and isopropanol, which are usually used for cosmetic purposes, can be used as alcohols.

Accordingly, in a preferred embodiment the agent additionally has at least one alcohol having 2 to 6 carbon atoms and 1 to 3 hydroxyl groups. This additional alcohol is again preferably chosen from at least one compound of ethanol, ethylene glycol, isopropanol, 1,2-propylene glycol, 1,3-propylene glycol, glycerin, n-butanol, 1,3-butylene glycol. A quite particularly preferred alcohol is ethanol.

The agent preferably comprises the additional alcohol having 2 to 6 carbon atoms and 1 to 3 hydroxyl groups (particularly in the presence of at least one propellant) in an amount of 40 wt % to 65 wt %, particularly 40 wt % to 50 wt %, based on weight of the cosmetic agent.

Organic solvents or mixture of solvents with a boiling point of less than 400° C. can be used as additional co-solvents in an amount of 0.1 to 15 wt %, preferably 1 to 10 wt %, based on total agent. Particularly suitable additional co-solvents are unbranched or branched hydrocarbons such as pentane, hexane, isopentane and cyclic hydrocarbons such as cyclopentane and cyclohexane. Additional, particularly preferred water-soluble solvents are glycerin, ethylene glycol and propylene glycol in an amount of up to 30 weight percent based on total agent.

In particular, the addition of glycerin and/or propylene glycol and/or polyethylene glycol and/or polypropylene glycol increases the flexibility of the polymer film that is formed when the agent according to the invention is used. Consequently, if a more flexible hold is desired, then the agents preferably comprise 0.01 to 30 wt % glycerin and/or propylene glycol and/or polyethylene glycol and/or polypropylene glycol, based on total agent.

The agents preferably exhibit a pH of 2 to 11. The pH range is particularly preferably from 2 to 8. In the context of this publication, pH data refer to the pH at 25° C. unless otherwise stated.

The inventive effects were increased by addition of at least one (C₂ to C₆) trialkyl citrate to the agent. Consequently, it is inventively preferred when the agents additionally comprise at least one compound of Formula E,

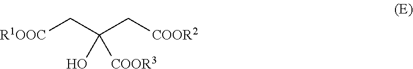

wherein R¹, R² and R³ independently of one another is a (C₂ to C₆) alkyl group. Exemplary (C₂ to C₆) alkyl groups according to Formula (E) are methyl, ethyl, isopropyl, n-propyl, n-butyl, sec-butyl, isobutyl, tert-butyl, n-pentyl, neopentyl, isopentyl, n-hexyl.

Triethyl citrate is a particularly preferred compound of Formula (E).

The agent preferably contains the compound of Formula (E) in an amount of 0.01 to 1 wt %, particularly 0.05 to 0.3 wt %, based on weight of the total agent.

A similar increase in the inventive effect can be achieved by adding isopropyl myristate. The agent preferably comprises this ester in an amount of 0.1 wt % to 1 wt %, particularly 0.05 wt % to 0.3 wt %, based on weight of the total agent.

In order to intensify the effect according to the invention, the agents preferably additionally comprise at least one surfactant, wherein in principal, non-ionic, anionic, cationic, ampholytic surfactants are suitable. The group of the ampholytic or also amphoteric surfactants includes zwitterionic surfactants and ampholytes. According to the invention, the surfactants can already have an emulsifying action. The addition of at least one non-ionic surfactant and/or at least one cationic surfactant is preferred in the context of this embodiment of the invention.

The agent preferably comprises additional surfactants in an amount of 0.01 wt % to 5 wt %, particularly preferably 0.05 wt % to 0.5 wt %, based on weight of the agent.

It has proved particularly preferable when the agents additionally comprise at least one non-ionic surfactant.

Non-ionic surfactants comprise, for example, a polyol group, a polyalkylene glycol ether group or a combination of polyol ether groups and polyglycol ether groups as the hydrophilic group. Exemplary compounds of this type are
addition products of 2 to 100 moles ethylene oxide and/or 1 to 5 moles propylene oxide to linear and branched fatty alcohols containing 8 to 30 carbon atoms, to fatty acids containing 8 to 30 carbon atoms and to alkyl phenols containing 8 to 15 carbon atoms in the alkyl group,
methyl or C₂-C₆ alkyl group end blocked addition products of 2 to 50 moles ethylene oxide and/or 1 to 5 moles propylene oxide to linear and branched fatty alcohols with 8 to 30 carbon atoms, to fatty acids with 8 to 30 carbon atoms and to alkyl phenols with 8 to 15 carbon atoms in the alkyl group, such as the commercially available types Dehydrol® LS, Dehydrol® LT (Cognis),
C₁₂-C₃₀ fatty acid mono and diesters of addition products of 1 to 30 moles ethylene oxide to glycerin,
addition products of 5 to 60 moles ethylene oxide on castor oil and hydrogenated castor oil,
polyol esters of fatty acids, such as, for example, the commercial product Hydagen® HSP (Cognis) or Sovermol types (Cognis),
alkoxylated triglycerides,
alkoxylated fatty acid alkyl esters of Formula (T-I)

R¹CO—(OCH₂CHR²)_{w}OR³ (T-I)

wherein R¹CO is a linear or branched, saturated and/or unsaturated acyl group containing 6 to 22 carbon atoms, R² is hydrogen or methyl, R³ is linear or branched alkyl groups containing 1 to 4 carbon atoms and w is a number from 1 to 20,
amine oxides,
mixed hydroxy ethers, such as are described for example in DE-OS 1 973 8866,
sorbitol esters of fatty acids and addition products of ethylene oxide to sorbitol esters of fatty acids such as the polysorbates,
sugar esters of fatty acids and addition products of ethylene oxide to sugar esters of fatty acids,
addition products of ethylene oxide to fatty acid alkanolamides and fatty amines,
sugar surfactants of alkyl and alkenyl oligoglycosides type according to Formula (T-II),

R⁴O-[G]ₚ (T-II)
wherein R⁴ is an alkyl or alkenyl group containing 4 to 22 carbon atoms, G is a sugar group containing 5 or 6 carbon atoms, and p is a number from 1 to 10. They can be obtained according to appropriate methods of preparative organic chemistry.

Alkylene oxide addition products to saturated, linear fatty alcohols and fatty acids, each with 2 to 100 moles ethylene oxide per mole fatty alcohol or fatty acid, have proven to be quite particularly preferred non-ionic surfactants. Similarly, preparations with excellent properties are obtained when they comprise C₁₂-C₃₀ fatty acid mono and diesters of addition products of 1 to 30 moles ethylene oxide to glycerin and/or addition products of 5 to 60 moles ethylene oxide to castor oil and hydrogenated castor oil as the non-ionic surfactants.

For surfactants, which are represented by the addition products of ethylene oxide and/or propylene oxide to fatty alcohols or derivatives of these addition products, both products with a “normal” homologue distribution as well as those with a narrow homologue distribution may be used. The term “normal” homologue distribution refers to mixtures of homologues obtained from the reaction of fatty alcohols and alkylene oxide using alkali metals, alkali metal hydroxides or alkali metal alkoxides as catalysts. Narrow homologue distributions are obtained if, for example, hydrotalcite, alkaline earth metal salts of ether carboxylic acids, alkaline earth metal oxides, hydroxides or alkoxides are used as the catalysts. Use of products with a narrow homologue distribution can be preferred.

Agents according to the invention quite particularly preferably comprise as the surfactant at least one addition product of 15 to 100 moles ethylene oxide, especially 15 to 50 moles ethylene oxide on a linear or branched (especially linear) fatty alcohol containing 8 to 22 carbon atoms. These are quite particularly preferably Ceteareth-15, Ceteareth-25 or Ceteareth-50, marketed as Eumulgin® CS 15 (COGNIS), Cremophor A25 (BASF SE) or Eumulgin® CS 50 (COGNIS).

Suitable anionic surfactants include all anionic surface-active materials suitable for use on the human body. They have a water solubilizing anionic group such as a carboxylate, sulfate, sulfonate or phosphate group, and a lipophilic alkyl group containing about 8 to 30 carbon atoms. In addition, the molecule may comprise glycol or polyglycol ether groups, ester, ether and amide groups as well as hydroxyl groups. Exemplary suitable anionic surfactants are, each in the form of the sodium, potassium and ammonium, as well as mono-, di- and trialkanolammonium salts containing 2 to 4 carbon atoms in the alkanol group,
linear and branched fatty acids with 8 to 30 carbon atoms (soaps),
ether carboxylic acids of Formula R—O—(CH₂—CH₂)ₓ—CH₂—COOH, wherein R is a linear alkyl group with 8 to 30 carbon atoms and x=0 or 1 to 16,
acyl sarcosides with 8 to 24 carbon atoms in the acyl group,
acyl taurides with 8 to 24 carbon atoms in the acyl group,
acyl isethionates with 8 to 24 carbon atoms in the acyl group,
mono- and dialkyl esters of sulfosuccinic acid with 8 to 24 carbon atoms in the alkyl group and mono-alkyl polyoxyethyl esters of sulfosuccinic acid with 8 to 24 carbon atoms in the alkyl group and 1 to 6 oxyethylene groups,
linear alkane sulfonates containing 8 to 24 carbon atoms,
linear alpha-olefin sulfonates containing 8 to 24 carbon atoms,
alpha-sulfo fatty acid methyl esters of fatty acids containing 8 to 30 carbon atoms,
alkyl sulfates and alkyl polyglycol ether sulfates of Formula R—O(CH₂—CH₂O)ₓ—OSO₃H wherein R is preferably a linear alkyl group having 8 to 30 carbon atoms and x=0 or 1 to 12,
mixtures of surface-active hydroxysulfonates,
sulfated hydroxyalkyl polyethylene glycol ethers and/or hydroxyalkylene propylene glycol ethers,
sulfonates of unsaturated fatty acids with 8 to 24 carbon atoms and 1 to 6 double bonds,
esters of tartaric acid and citric acid with alcohols, representing the addition products of about 2-15 molecules of ethylene oxide and/or propylene oxide on fatty alcohols containing 8 to 22 carbon atoms,
alkyl- and/or alkenyl ether phosphates of Formula (T-V)

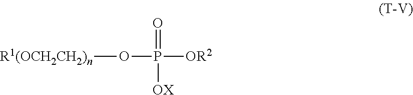

wherein R¹ preferably is an aliphatic hydrocarbon group containing 8 to 30 carbon atoms, R² is hydrogen, a (CH₂CH₂O)ₙR group or X, n is a number from 1 to 10, and X is hydrogen, an alkali metal or alkaline earth metal or NR³R⁴R⁵R⁶, with R³ to R⁶, independently of each other, standing for a C₁ to C₄ hydrocarbon group,
sulfated fatty acid alkylene glycol esters of Formula (T-VI)

R⁷CO(AlkO)ₙSO₃M (T-VI)
wherein R⁷CO is a linear or branched, aliphatic, saturated and/or unsaturated acyl group with 6 to 22 carbon atoms, Alk is CH₂CH₂, CHCH₃CH₂ and/or CH₂CHCH₃, n is a number from 0.5 to 5, and M is a cation,
monoglyceride sulfates and monoglyceride ether sulfates of Formula (T1-VII)

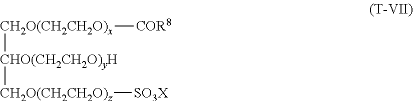

wherein R⁸CO is a linear or branched acyl group containing 6 to 22 carbon atoms, the sum of x, y and z is 0 or a number from 1 to 30, preferably 2 to 10, and X is an alkali metal or alkaline earth metal. Preferably, monoglyceride sulfates of Formula (T-VII) are employed wherein R⁸CO is a linear acyl group containing 8 to 18 carbon atoms,
amide ether carboxylic acids,
condensation products of C₈-C₃₀ fatty alcohols with protein hydrolyzates and/or amino acids and their derivatives, known to one skilled in the art as albumin fatty acid condensates, such as the Lamepon® types, Gluadin® types, Hostapon® KCG or the Amisoft® types.

Preferred anionic surfactants are alkyl sulfates, alkyl polyglycol ether sulfates and ether carboxylic acids with 10 to 18 carbon atoms in the alkyl group and up to 12 glycol ether groups in the molecule, sulfosuccinic acid mono and dialkyl esters with 8 to 18 C atoms in the alkyl group and sulfosuccinic acid mono-alkyl polyoxyethyl esters with 8 to 18 C atoms in the alkyl group and 1 to 6 oxyethylene groups, monoglycerin disulfates, alkyl and alkenyl ether phosphates as well as albumin fatty acid condensates.

According to the invention, cationic surfactants of the type quaternary ammonium compounds, the esterquats and the amido amines can likewise be used. Preferred quaternary ammonium compounds are ammonium halides, especially chlorides and bromides such as alkyl-trimethylammonium chlorides, dialkyldimethylammonium chlorides and trialkylmethylammonium chlorides. The long alkyl chains of these surfactants preferably have 10 to 18 carbon atoms, such as in cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. Further preferred cationic surfactants are those imidazolium compounds known under the INCI names Quaternium-27 and Quaternium-83.

Zwitterionic surfactants are those surface-active compounds having at least one quaternary ammonium group and at least one —COO⁽⁻⁾ or —SO₃⁽⁻⁾ group in the molecule. Particularly suitable zwitterionic surfactants are betaines such as the N-alkyl-N,N-dimethylammonium glycinates, for example, cocoalkyl-dimethylammonium glycinate, N-acyl-aminopropyl-N,N-dimethylammonium glycinate, for example, coco-acylaminopropyl-dimethylammonium glycinate, and 2-alkyl-3-carboxymethyl-3-hydroxyethyl-imidazolines each with 8 to 18 carbon atoms in the alkyl or acyl group as well as the cocoacyl-aminoethylhydroxyethylcarboxymethyl glycinate. A preferred zwitterionic surfactant is the fatty acid amide derivative known under the INCI name Cocamidopropyl Betaine.

Ampholytes include such surface-active compounds that apart from a C₈₋₂₄ alkyl or acyl group, comprise at least one free amino group and at least one —COOH or —SO₃H group in the molecule and are able to form internal salts. Examples of suitable ampholytes are N-alkylglycines, N-alkyl propionic acids, N-alkylamino butyric acids, N-alkylimino dipropionic acids, N-hydroxyethyl-N-alkylamidopropylglycines, N-alkyltaurines, N-alkylsarcosines, 2-alkylamino propionic acids and alkylamino acetic acids, each with about 8 to 24 carbon atoms in the alkyl group. Particularly preferred ampholytes are N-cocoalkylamino propionate, cocoacylaminoethylamino propionate and C₁₂-C₁₈ acyl sarcosine.

Agents according to the invention can also have at least one amphoteric polymer as the film-forming and/or setting polymer. These additional polymers differ from the previously defined polyamides (a) and the previously defined amphoteric polymers (b).

Film-forming polymers refer to those polymers that on drying leave a continuous film on the skin, the hair or the nails. These types of film-former can be used in the widest variety of cosmetic products such as make up masks, make up, hair sets, hair sprays, hair gels, hair waxes, hair conditioners, shampoos or nail varnishes. Those polymers are particularly preferred which are sufficiently soluble in alcohol or water/alcohol mixtures, such that they are present in completely dissolved form in the agent. Film-forming polymers can be of synthetic or of natural origin.

According to the invention, film-forming polymers are further understood to mean those polymers that, when used in concentrations of 0.01 to 20 wt % in aqueous, alcoholic or aqueous alcoholic solution, are able to precipitate out a transparent polymer film on the hair.

Setting polymers contribute to the hold and/or creation of hair volume and hair body of the whole hairstyle. These polymers are also film-forming polymers at the same time and therefore, in general, are typical substances for styling hair treatment agents such as hair sets, hair foams, hair waxes, hair sprays. Film formation can be in completely selected areas and bond only some fibers together.

The curl-retention test is frequently used as a test method for the setting action.

Further, the agent according to the invention can have at least one film-forming cationic and/or setting cationic polymer.

The additional film-forming cationic and/or setting cationic polymers preferably possess at least one structural unit having at least one permanently cationized nitrogen atom. Permanently cationized nitrogen atoms refer to those nitrogen atoms having a positive charge and thereby form a quaternary ammonium compound. Quaternary ammonium compounds are mostly produced by reacting tertiary amines with alkylating agents, such as methyl chloride, benzyl chloride, dimethyl sulfate, dodecyl bromide, as well as ethylene oxide. Depending on the tertiary amine, the following groups are particularly well known: alkylammonium compounds, alkenylammonium compounds, imidazolinium compounds and pyridinium compounds.

The agent according to the invention preferably has at least one film-forming and/or setting polymer that is preferably chosen from at least one polymer from non-ionic polymers, cationic polymers, amphoteric polymers, zwitterionic polymers and anionic polymers.

The agent preferably has film-forming and/or setting polymers in an amount of 0.01 wt % to 20.0 wt %, more preferably 0.5 wt % to 15 wt %, quite particularly preferably 2.0 wt % to 10.0 wt %, based on total weight of the agent. These quantitative data also apply for all subsequent preferred types of film-forming and/or setting polymers that can be used in the inventive agents. Should subsequently different preferred quantities be specified, then the latter are to be again taken as the preferred quantities.

Those agents are particularly preferably suitable that have at least one film-forming and/or setting polymer chosen from at least one polymer of—
non-ionic polymers based on ethylenically unsaturated monomers, in particular, from
   homopolymers of N-vinyl pyrrolidone,
   non-ionic copolymers of N-vinyl pyrrolidone,
   homopolymers and non-ionic copolymers of N-vinyl caprolactam,
   copolymers of (meth)acrylamide,
   polyvinyl alcohol, polyvinyl acetate,
chitosan and derivatives of chitosan,
cationic cellulose derivatives,
cationic copolymers of 3-(C₁ to C₆) alkyl-1-vinyl-imidazolinium,
homopolymers and copolymers comprising the structural unit of Formula (M−1)

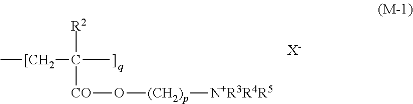

wherein R²═—H or —CH₃, R³, R⁴ and R⁵ independently of each other are chosen from (C₁ to C₄) alkyl, (C₁ to C₄) alkenyl or (C₂ to C₄) hydroxyalkyl groups, p=1, 2, 3 or 4, q is a natural number, and X⁻ is a physiologically acceptable organic or inorganic anion,
anionic polymers that exhibit carboxylate and/or sulfonate groups,
anionic polyurethanes.

Preferred non-ionic polymers, based on ethylenically unsaturated monomers, which are suitable as additional film-forming and/or setting polymers are those non-ionic polymers that comprise at least one of the following structural units—

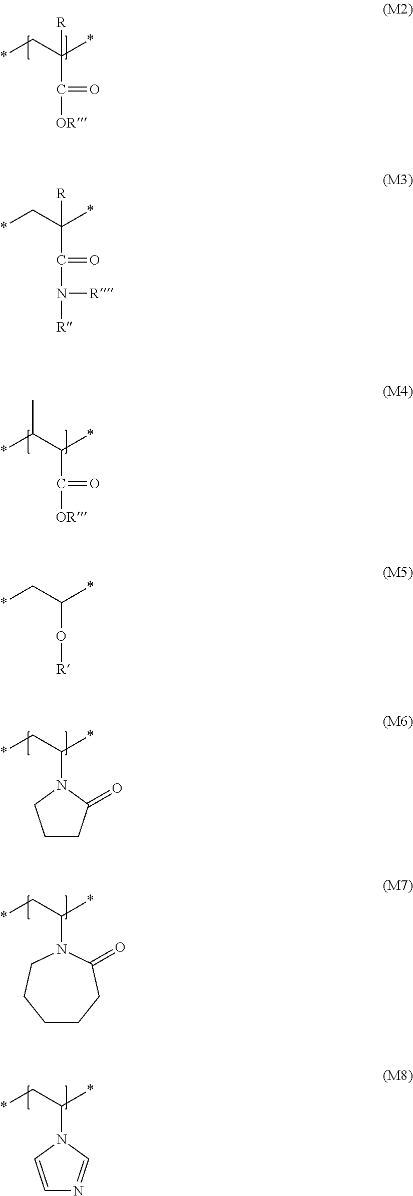

wherein
R is a hydrogen atom or a methyl group,
R′ is a hydrogen atom or a (C₁ to C₄) acyl group,
R″ and R′″ independently of one another are a (C₁ to C₇) alkyl group or a hydrogen atom,
R′″ is a linear or branched (C₁ to C₄) alkyl group or a (C₂ to C₄) hydroxyalkyl group.

Suitable, non-ionic film-forming and/or non-ionic hair setting polymers are homopolymers or copolymers based on at least one of the following monomers: vinyl pyrrolidone, vinyl caprolactam, vinyl esters such as vinyl acetate, vinyl alcohol, acrylamide, methacrylamide, alkyl and dialkyl acrylamide, alkyl and dialkyl methacrylamide, alkyl acrylate, alkyl methacrylate, wherein each of the alkyl groups of these monomers are chosen from (C₁ to C₃) alkyl groups.

For agents according to the invention, particularly suitable non-ionic polymers based on ethylenically unsaturated monomers have at least one of the following structural units—

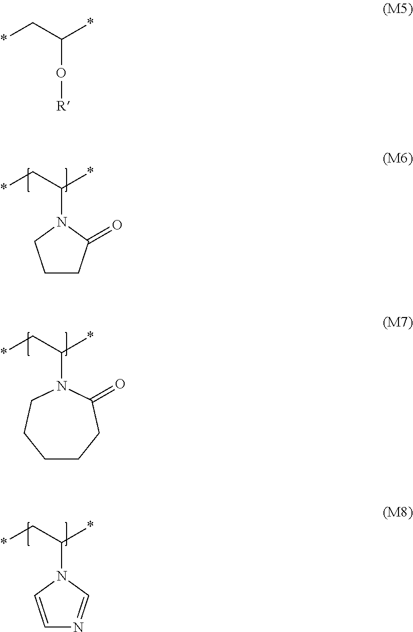

wherein R′ is a hydrogen atom or a (C₁ to C₃₀) acyl group, particularly a hydrogen atom or an acetyl group.

Homopolymers of vinyl caprolactam or vinyl pyrrolidone (such as Luviskol® K 90 or Luviskol® K 85 from BASF SE), copolymers of vinyl pyrrolidone and vinyl acetate (such as are marketed under the trade names Luviskol® VA 37, Luviskol® VA 55, Luviskol® VA 64 and Luviskol® VA 73 by BASF SE), terpolymers of vinyl pyrrolidone, vinyl acetate and vinyl propionate, polyacrylamides (such as Akypomine® P 191 from CHEM-Y), polyvinyl alcohols (marketed, for example, under the trade names Elvanol® by Du Pont or Vinci® 523/540 by Air Products), terpolymers of vinyl pyrrolidone, methacrylamide and vinyl imidazole (such as Luviset® Clear from BASF SE) are particularly suitable.

In addition to non-ionic polymers based on ethylenically unsaturated monomers, non-ionic cellulose derivatives are also suitable film-forming and/or setting polymers for the preferred achievement of the technical teaching. They are preferably chosen from methyl cellulose, especially from cellulose ethers such as hydroxypropyl cellulose (e.g., hydroxypropyl cellulose with a molecular weight of 30,000 to 50,000 g/mol, marketed, for example, under the trade name Nisso SI® by Lehmann & Voss, Hamburg), hydroxyethyl celluloses, such as are marketed under the trade names Culminal® and Benecel® (AQUALON) and Natrosol® types (Hercules).

Cationic polymers refer to polymers that, in their main chain and/or side chain, possess groups that can be “temporarily” or “permanently” cationic. “Permanently cationic” refers, according to the invention, to those polymers having a cationic group, independently of the pH of the medium. These are generally polymers having, for example, a quaternary nitrogen atom in the form of an ammonium group. Preferred cationic groups are quaternary ammonium groups. In particular, those polymers wherein the quaternary ammonium groups are bonded through a C₁₋₄ hydrocarbon group to a polymer backbone formed from acrylic acid, methacrylic acid or their derivatives have proved to be particularly suitable.

An inventively preferred suitable cationic film-forming and/or cationic setting polymer is at least one cationic film-forming and/or cationic setting polymer having at least one structural element of Formula (M9) and additionally at least one structural element of Formula (M10)—

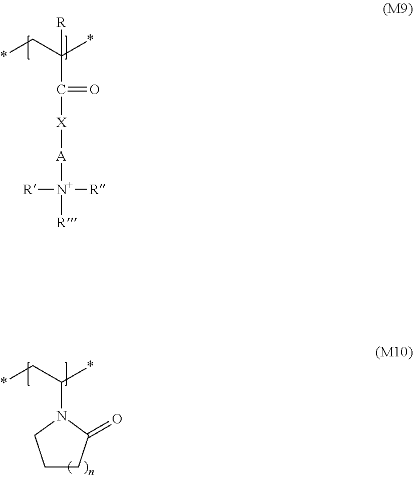

wherein
R is a hydrogen atom or a methyl group,
R′, R″ and R′″ are, independently of one another, a (C₁ to C₃₀) alkyl group,
X is an oxygen atom or an NH group,
A is an ethane-1,2-diyl group or a propane-1,3-diyl group,
n is 1 or 3.

To compensate for the positive polymer charge, all possible physiologically acceptable anions may be used, such as chloride, bromide, hydrogen sulfate, methyl sulfate, ethyl sulfate, tetrafluoroborate, phosphate, hydrogen phosphate, dihydrogen phosphate or p-toluene sulfonate, triflate. Exemplary compounds of this type are—
Copolymers of dimethylaminoethyl methacrylate, quaternized with diethyl sulfate, with vinyl pyrrolidone having the INCI name Polyquaternium-11 under the trade names Gafquat® 440, Gafquat® 734, Gafquat® 755 (each from ISP) and Luviquat PQ 11 PN (BASF SE),
Copolymers of N-vinyl pyrrolidone, N-vinyl caprolactam, N-(3-dimethylaminopropyl)methacrylamide and 3-(methacryloylamino)propyl-lauryl-dimethylammonium chloride (INCI name: Polyquaternium-69), commercially available, for example, under the trade name Aquastyle® 300 (28-32 wt % active substance in water/ethanol mixture) by the ISP Company.

Furthermore, the cationic film-forming and/or cationic setting polymers are inventively particularly preferably chosen from cationic, quaternized cellulose derivatives.

Moreover, cationic, quaternized cellulose derivatives are preferred suitable film-forming and/or setting polymers.

Those cationic, quaternized celluloses having more than one permanent cationic charge in a side chain have proven to be particularly advantageous in the context of the invention. Among these cationic celluloses, once again those cationic celluloses with the INCI name Polyquaternium-4 are particularly suitable which are marketed, for example, by the National Starch Company under the trade names Celquat® H 100, Celquat® L 200.

In the context of the invention, those cationic film-forming and/or cationic setting copolymers having at least one structural element of Formula (M11) additionally serve as particularly preferred usable cationic polymers—

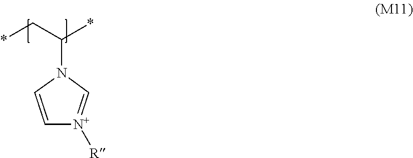

wherein R″ is a (C₁ to C₄) alkyl group, especially a methyl group, and additionally possesses at least one other cationic and/or non-ionic structural element.

To compensate for the positive polymer charge, all possible physiologically acceptable anions may be used, such as chloride, bromide, hydrogen sulfate, methyl sulfate, ethyl sulfate, tetrafluoroborate, phosphate, hydrogen phosphate, dihydrogen phosphate or p-toluene sulfonate, triflate.

It is again inventively preferred when at least one copolymer (c1) that, in addition to at least one structural element of Formula (M11), further contains a structural element of Formula (M6), is comprised as the additional cationic film-forming and/or cationic setting polymer—

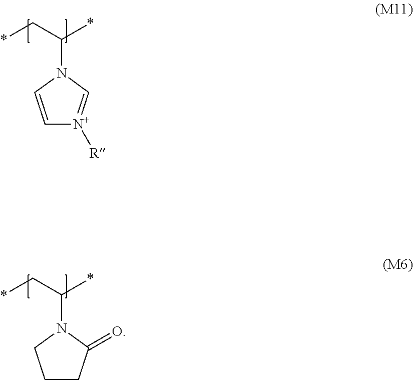

wherein R″ is a (C₁ to C₄) alkyl group, particularly a methyl group.

To compensate for the positive polymer charge of the copolymer (c1), all possible physiologically acceptable anions may be used, such as chloride, bromide, hydrogen sulfate, methyl sulfate, ethyl sulfate, tetrafluoroborate, phosphate, hydrogen phosphate, dihydrogen phosphate or p-toluene sulfonate, triflate.

Cationic film-forming and/or cationic setting polymers that are quite particularly preferred as copolymers (c1) have 10 to 30 mol %, preferably 15 to 25 mol % and particularly 20 mol % of structural units according to Formula (M11) and 70 to 90 mol %, preferably 75 to 85 mol % and particularly 80 mol % of structural units according to Formula (M6).

Here, it is particularly preferred when copolymers (c1) comprise, in addition to polymer units resulting from incorporation of the cited structural units according to Formula (M11) and (M6) into the copolymer, maximum 5 wt %, preferably maximum 1 wt % of polymer units that trace back to the incorporation of other monomers. Copolymers (c1) are preferably exclusively constructed of structural units of Formula (M11) with R″=methyl and (M6).

If a chloride ion is used to compensate the positive charge of the polymer of Formula (Polyl), then according to INCI nomenclature these N-methylvinyl imidazole/vinyl pyrrolidone copolymers are named Polyquaternium-16 and are available, for example, under the trade names Luviquat® Style, Luviquat® FC 370, Luviquat® FC 550, Luviquat® FC 905 and Luviquat® HM 552 from BASF.

If a methosulfate ion is used to compensate the positive charge of the polymer of Formula (Polyl), then according to INCI nomenclature these N-methylvinyl imidazole/vinyl pyrrolidone copolymers are named Polyquaternium-44 and are available, for example, under the trade names Luviquat® UltraCare from BASF.

Particularly preferred inventive compositions comprise a copolymer (c1) having molecular masses within a defined range. Here, inventive agents are preferred wherein the molecular mass of copolymer (c1) is from 50 to 400 kDa, preferably from 100 to 300 kDa, more preferably from 150 to 250 kDa and particularly from 190 to 210 kDa.

In addition to copolymer(s) (c1) or instead of it or them, the inventive agents can also comprise copolymers (c2) that starting from the copolymer (c1) possess structural units of Formula (M7) as the additional structural units—

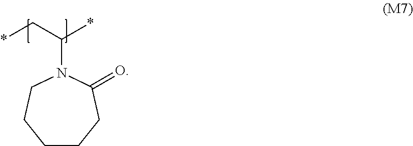

Further particularly preferred agents according to the invention are accordingly those having as the cationic film-forming and/or cationic setting polymer at least one copolymer (c2) having at least one structural unit according to Formula (M11-a), at least one structural unit according to Formula (M6), and at least one structural unit according to Formula (M7)—

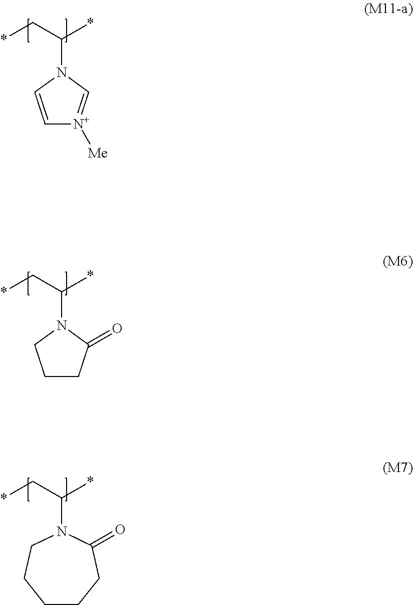

Also in this regard it is particularly preferred when copolymers (c2) comprise, in addition to polymer units resulting from incorporation of the cited structural units according to Formula (M11-a), (M6) and (M7) into the copolymer, maximum 5 wt %, preferably maximum 1 wt % of polymer units that trace back to the incorporation of other monomers. Copolymers (c2) are preferably exclusively constructed from structural units of Formulas (M11-a), (M6) and (M7).

To compensate for the positive polymer charge of component (c2), all possible physiologically acceptable anions can be used, such as chloride, bromide, hydrogen sulfate, methyl sulfate, ethyl sulfate, tetrafluoroborate, phosphate, hydrogen phosphate, dihydrogen phosphate or p-toluene sulfonate, triflate.

If a methosulfate ion is used to compensate the positive charge of the polymer of Formula (Poly2), then according to INCI nomenclature these N-methylvinyl imidazole/vinyl pyrrolidone/vinyl caprolactam copolymers are named Polyquaternium-46 and are available for example under the trade names Luviquat® Hold from BASF.

Quite particularly preferred copolymers (c2) comprise 1 to 20 mol %, preferably 5 to 15 mol % and particularly 10 mol % of structural units in accordance with Formula (M11-a) and 30 to 50 mol %, preferably 35 to 45 mol % and particularly 40 mol % of structural units in accordance with Formula (M6) and 40 to 60 mol %, preferably 45 to 55 mol % and particularly 60 mol % of structural units in accordance with Formula (M7).

Particularly preferred inventive agents comprise a copolymer (c2) having molecular masses within a defined range. Here, inventive agents are preferred wherein the molecular mass of copolymer (c2) is from 100 to 1000 kDa, preferably from 250 to 900 kDa, more preferably from 500 to 850 kDa and particularly from 650 to 710 kDa.

In addition to copolymer(s) (c1) and/or (c2) or in its or their place, agents according to the invention can also have copolymers (c3) as the film-forming cationic and/or setting cationic polymer which possess as the structural units structural units of Formulas (M11-a) and (M6), as well as additional structural units from the group of vinyl imidazole units and further structural units from the group of acrylamide and/or methacrylamide units.

Further particularly preferred agents according to the invention comprise as the additional cationic film-forming and/or cationic setting polymer at least one copolymer (c3) having at least one structural unit according to Formula (M11-a), at least one structural unit according to Formula (M6), at least one structural unit according to Formula (M10) and at least one structural unit according to Formula (M12)—

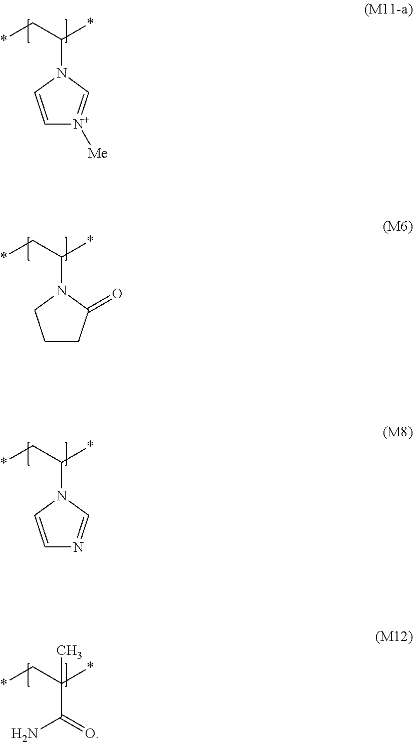

Also in this regard it is particularly preferred when copolymers (c3) have, in addition to polymer units resulting from incorporation of the cited structural units according to Formula (M11-a), (M6), (M8) and (M12) into the copolymer, a maximum of 5 wt %, preferably a maximum of 1 wt % of polymer units that trace back to the incorporation of other monomers. Copolymers (c3) are preferably exclusively constructed from structural units of Formulas (M11-a), (M6), (M8) and (M12).

To compensate for the positive polymer charge of component (c3), all possible physiologically acceptable anions can be used, such as chloride, bromide, hydrogen sulfate, methyl sulfate, ethyl sulfate, tetrafluoroborate, phosphate, hydrogen phosphate, dihydrogen phosphate or p-toluene sulfonate, triflate.

If a methosulfate ion is used to compensate the positive charge of the polymer of Formula (Poly3), then according to INCI nomenclature these N-methylvinyl imidazole/vinyl pyrrolidone/vinyl imidazole/methacrylamide copolymers are named Polyquaternium-68 and are available for example under the trade names Luviquat® Supreme from BASF.

Quite particularly preferred copolymers (c3) comprise 1 to 12 mol %, preferably 3 to 9 mol % and particularly 6 mol % of structural units according to Formula (M11-a) and 45 to 65 mol %, preferably 50 to 60 mol % and particularly 55 mol % of structural units according to Formula (M6) and 1 to 20 mol %, preferably 5 to 15 mol % and particularly 10 mol % of structural units according to Formula (M8) and 20 to 40 mol %, preferably 25 to 35 mol % and particularly 29 mol % of structural units according to Formula (M12).

Particularly preferred inventive agents comprise a copolymer (c3) having molecular masses within a defined range. Here, inventive agents are preferred wherein the molecular mass of copolymer (c3) is from 100 to 500 kDa, preferably from 150 to 400 kDa, more preferably from 250 to 350 kDa, and particularly from 290 to 310 kDa.

Preferred additional film-forming cationic and/or setting polymers, chosen from cationic polymers with at least one structural element of the above Formula (M11-a), include:
vinyl pyrrolidone/1-vinyl-3-methyl-1H-imidazolium chloride copolymers (such as that with the INCI name Polyquaternium-16, sold under the trade names Luviquat® Style, Luviquat® FC 370, Luviquat® FC 550, Luviquat® FC 905 and Luviquat® HM 552 (BASF SE)),
vinyl pyrrolidone/1-vinyl-3-methyl-1H-imidazolium methyl sulfate copolymers (such as that with the INCI name Polyquaternium-44 sold under the trade name Luviquat® Care (BASF SE)),
vinyl pyrrolidone/vinyl caprolactam/1-vinyl-3-methyl-1H-imidazolium terpolymer (such as that with the INCI name Polyquaternium-46 sold under the trade names Luviquat® Care or Luviquat® Hold (BASF SE)),
vinyl pyrrolidone/methacrylamide/vinyl imidazole/1-vinyl-3-methyl-1H-imidazolium methyl sulfate copolymer (such as that with the INCI name Polyquaternium-68 sold under the trade name Luviquat® Supreme (BASF SE)),
   as well as mixtures of these polymers.

Further preferred cationic polymers that can be employed in the inventive agents are the “temporarily cationic” polymers. These polymers usually comprise an amino group that is present at specific pH values as a quaternary ammonium group and is thus cationic.

These polymers include, for example, chitosan. In the present invention, chitosan and/or chitosan derivatives are considered as quite particularly preferred suitable film-forming and/or setting polymers.

Chitosans are biopolymers and are hydrocolloids. From the chemical point of view, they are partially deacetylated chitins of different molecular weight.

Chitosan is manufactured from chitin, preferably from the remains of crustacean shells, which are available in large quantities as a cheap raw material. The chitin is usually deprotonated by addition of bases, demineralized by adding mineral acids and finally deacetylated by adding strong bases, wherein the molecular weights can vary over a broad spectrum. Those types are preferably employed that have an average molecular weight of 800,000 to 1,200,000 Dalton, a Brookfield viscosity (1% conc. in glycolic acid) below 5000 mPas, a deacetylation degree in the range of 80 to 88%, and an ash content of less than 0.3 wt %.

In the scope of the invention, besides chitosans as the typical cationic biopolymers, cationically derivatized chitosans (e.g., quaternized products) or alkoxylated chitosans can also be considered.

Inventively preferred agents comprise neutralization products of chitosan neutralized with at least one acid chosen from lactic acid, pyrrolidone carboxylic acid, nicotinic acid, hydroxy-iso-butyric acid, hydroxy-iso-valeric acid, or contain mixtures of these neutralization products as the chitosan derivative(s).

Exemplary suitable chitosan (derivatives) are freely available on the market under the trade names Hydagen® CMF (1 wt % active substance in aqueous solution with 0.4 wt % glycolic acid, molecular weight 500 000 to 5 000 000 g/mol Cognis), Hydagen® HCMF (chitosan (80% deacetylated), molecular weight 50 000 to 1 000 000 g/mol, Cognis), Kytamer® PC (80 wt % active substance of chitosan pyrrolidone carboxylate (INCI name: Chitosan PCA), Amerchol) and Chitolam® NB/101.

The agents preferably contain chitosan or its derivatives in an amount of 0.01 wt % to 20.0 wt %, more preferably 0.01 wt % to 10.0 wt %, quite particularly preferably 0.1 wt % to 1 wt %, based on total weight of the agent according to the invention.

In the context of the invention, preferred suitable temporarily cationic polymers are likewise those having at least one structural unit of Formulas (M1-1) to (M1-8)

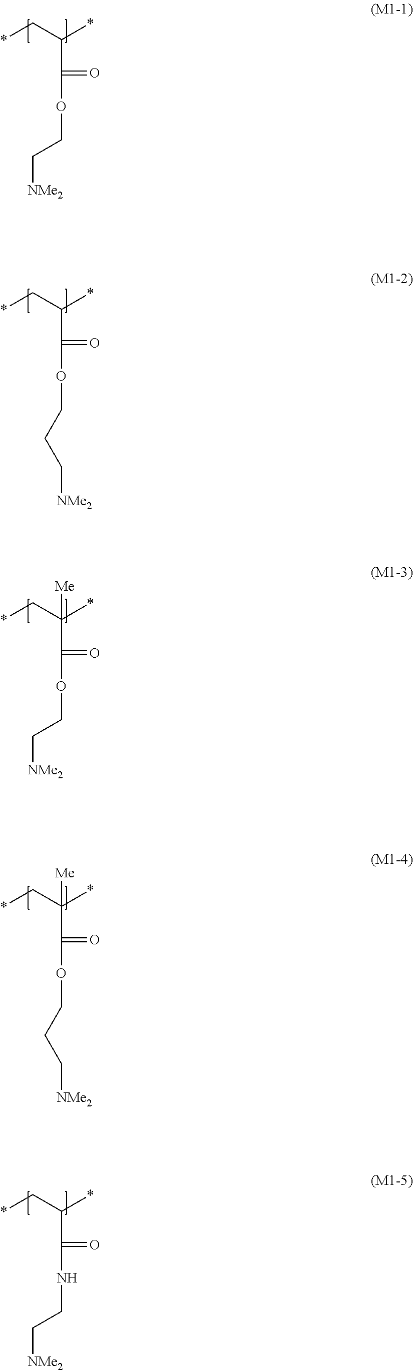

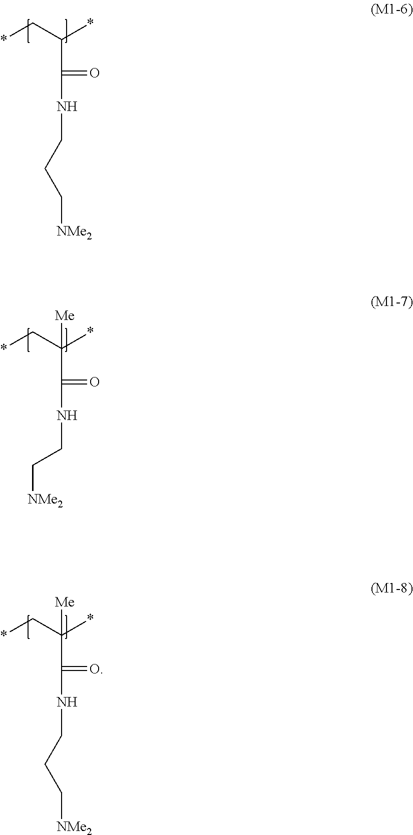

In this regard, those copolymers are again preferred that have at least one structural unit of Formulas (M1-1) to (M1-8) and in addition at least one structural unit of Formula (M10)

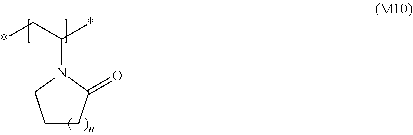

wherein n is 1 or 3.

Here again, the group of polymers—
vinyl caprolactam/vinyl pyrrolidone/dimethylaminoethyl methacrylate copolymer (e.g., INCI name: Vinyl Caprolactam/PVP/Di-methylaminoethyl Methacrylate Copolymer under the trade name Gaffix® VC 713 (ISP)),
N-vinyl pyrrolidone/N-vinyl caprolactam/dimethylaminopropylmethacrylamide copolymer (e.g., INCI name: VPNinyl Caprolactam/DMAPA Acrylates Copolymer under the trade name Aquaflex® SF-40 (ISP)),
vinyl caprolactam/vinyl pyrrolidone/dimethylaminoethyl methacrylate copolymer (e.g., as a 35-39% solids in ethanol in the form of the commercial product Advantage LC E with the INCI name: Vinyl CaprolactamNP/Dimethylaminoethyl Methacrylate Copolymer, Alcohol, Lauryl Pyrrolidone (ISP)),
vinyl pyrrolidone/dimethylaminopropylmethacrylamide copolymer (e.g., INCI name: VP/DMAPA Acrylates Copolymer under the trade name Styleze® CC-10 (ISP)), represent a preferred list from which at least one or more polymers can be chosen.

Agents according to the invention can also have at least one amphoteric polymer as the film-forming and/or setting polymer. The term amphopolymers includes not only those polymers whose molecule has both free amino groups and free —COOH or SO3H groups and which are capable of forming inner salts, but also zwitterionic polymers whose molecule has quaternary ammonium groups and —COO⁻ or —SO₃⁻ groups, and polymers comprising —COOH or SO₃H groups and quaternary ammonium groups.

The agents preferably contain amphoteric polymers in amounts of 0.01 to 20 wt %, more preferably 0.05 to 10 wt %, based on total agent. Quantities of 0.1 to 5.0 wt % are quite particularly preferred.

Furthermore, at least one anionic film-forming and/or anionic setting polymer can be employed as the film-forming and/or setting polymers.

Anionic polymers concern anionic polymers having carboxylate and/or sulfonate groups. Exemplary anionic monomers from which such polymers can be made are acrylic acid, methacrylic acid, crotonic acid, maleic anhydride and 2-acrylamido-2-methylpropane sulfonic acid. Here, the acidic groups may be fully or partially present as sodium, potassium, ammonium, mono- or triethanolammonium salts.

Within this embodiment, it can be preferred to use copolymers of at least one anionic monomer and at least one non-ionic monomer. Regarding the anionic monomers, reference is made to the abovementioned substances. Preferred non-ionic monomers are acrylamide, methacrylamide, acrylic acid esters, methacrylic acid esters, vinyl pyrrolidone, vinyl ethers and vinyl esters.

Preferred anionic copolymers are acrylic acid-acrylamide copolymers, particularly polyacrylamide copolymers with monomers containing sulfonic acid groups. A particularly preferred anionic copolymer consists of 70 to 55 mole % acrylamide and 30 to 45 mole % 2-acrylamido-2-methylpropane sulfonic acid, wherein the sulfonic acid group may be fully or partially present as the sodium, potassium, ammonium, mono or triethanolammonium salt. This copolymer can also be crosslinked, wherein preferred crosslinking agents include polyolefinically unsaturated compounds such as tetraallyloxyethane, allyl sucrose, allyl pentaerythritol and methylene bisacrylamide. Such a polymer is found in the commercial product Sepigel®305 from the SEPPIC Company. Use of this compound, which comprises a mixture of hydrocarbons (C₁₃-C₁₄ isoparaffins) and a non-ionic emulsifier (Laureth-7) besides the polymer components, has proved to be particularly advantageous.

Sodium acryloyl dimethyl taurate copolymers commercialized as a compound with isohexadecane and polysorbate 80 under the trade name Simulgel®600 have also proved to be particularly effective according to the invention.

Likewise preferred anionic homopolymers are uncrosslinked and crosslinked polyacrylic acids. Here the preferred crosslinking agents can be allyl ethers of pentaerythritol, of sucrose and of propylene. Such compounds are commercially available, for example, under the trade name Carbopol®.

Further preferred employable anionic polymers are chosen from:
copolymers of vinyl acetate and crotonic acid (marketed, for example, as the commercial product Aristoflex® A 60 with the INCI name VA/Crotonates Copolymer by CIBA in a 60 wt. % conc. dispersion in isopropanol-water),
copolymers of ethyl acrylate and methacrylic acid (marketed, for example, under the trade name Luviflex® Soft with an acid number of 84 to 105 under the INCI name Acrylates Copolymer in a ca. 20 to 30 wt % conc. dispersion in water by BASF SE),
polyurethanes having at least one carboxylic group (such as a copolymer of isophthalic acid, adipic acid, 1,6-hexane diol, neopentyl glycol and isophorone diisocyanate, as marketed under the trade name Luviset® PUR with the INCI name Polyurethane-1 by BASF SE).

When particularly strong acting thickening anionic polymers are used, then in a preferred embodiment, care should be taken that the previously cited preferred viscosity criterion of the agent according to the invention is adhered to.

Copolymers of maleic anhydride and methyl vinyl ether, especially those with crosslinks are also color-conserving polymers. A maleic acid-methyl vinyl ether copolymer crosslinked with 1,9-decadiene is commercially available under the trade name Stabileze® QM.

The inventive agents can contain organic solvents or a mixture of solvents as additional co-solvents with a boiling point below 400° C. in an amount of 0.1 to 15 wt %, preferably 1 to 10 wt %, relative to the total agent. Unbranched or branched hydrocarbons such as pentane, hexane, isopentane and cyclic hydrocarbons such as cyclopentane and cyclohexane are particularly preferred as the additional co-solvent. Further, particularly preferred water-soluble solvents are glycerin, ethylene glycol and propylene glycol in an amount of up to 30 wt %, relative to the total agent.

In particular, the addition of glycerin and/or propylene glycol and/or polyethylene glycol increases the flexibility of the polymer film formed when the inventive agent is used. If a more flexible hold is required, the inventive agents preferably contain 0.01 to 30 wt % glycerin and/or propylene glycol and/or polyethylene glycol and/or polypropylene glycol relative to the total agent.

The pH of the agents is preferably from 2 to 11. A particularly preferred pH is from 2 to 8. In this document, pH values refer to the pH at 25° C., unless otherwise stated.

Agents according to the invention can additionally comprise auxiliaries and additives that are usually incorporated into conventional styling agents.

In particular, additional care products may be mentioned as suitable auxiliaries and additives.

The agent can have, for example, at least one protein hydrolyzate and/or one of its derivatives as a care substance.

Protein hydrolyzates are product mixtures obtained by acid-, base- or enzyme-catalyzed degradation of proteins (albumins). According to the invention, the term “protein hydrolyzates” also refers to total hydrolyzates as well as individual amino acids and their derivatives as well as mixtures of different amino acids. Furthermore, according to the invention, polymers built up from amino acids and amino acid derivatives are understood to be included in the term protein hydrolysates. The latter include polyalanine, polyasparagine, polyserine etc. Additional examples of usable compounds according to the invention are L-alanyl-L-proline, polyglycine, glycyl-L-glutamine or D/L-methionine-5-methylsulfonium chloride. Of course, (3-amino acids and their derivatives, like β-alanine, anthranilic acid or hippuric acid, can also be inventively added. The molecular weight of protein hydrolyzates utilizable according to the invention ranges from 75, the molecular weight of glycine, to 200,000, preferably the molecular weight is 75 to 50,000 and quite particularly preferably 75 to 20,000 Dalton.

According to the invention, the added protein hydrolyzates can be vegetal as well as animal, marine or synthetic origin.

Animal protein hydrolysates include protein hydrolysates of elastin, collagen, keratin, silk and milk albumin, which can also be present in the form of their salts. Such products are marketed, for example, under the trade names Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), Sericin (Pentapharm) and Kerasol® (Croda).

Use of silk protein hydrolyzates is particularly interesting. Silk refers to the fibers from the cocoon of the mulberry silk spinner (Bombyx mori L.). The raw silk fibers consist of a double stranded fibroin. Sericin is the intercellular cement that holds these double strands together. Silk consists of 70-80 wt % fibroin, 19-28 wt % sericin, 0.5-1 wt % fat and 0.5-1 wt % colorants and mineral constituents.

The major components of sericin are hydroxyamino acids with 46 wt %. Sericin consists of a group of 5 to 6 proteins. The major amino acids of sericin are serine (Ser, 37 wt %), aspartate (Asp, 26 wt %), glycine (Gly, 17 wt %), alanine (Ala), leucine (Leu) and tyrosine (Tyr).

The water-insoluble fibroin is counted as a sclero protein with a long chain molecular structure. The principle components of fibroin are glycine (44 wt %), alanine (26 wt %), and tyrosine (13 wt %). Another important structural feature of fibroin is the hexapeptide sequence Ser-Gly-Ala-Gly-Ala-Gly.

Technically, it is possible to easily separate both silk proteins from one another. Thus it is not surprising that sericin and fibroin are each individually known for use in cosmetic products. Furthermore, protein hydrolyzates and derivatives based on each of the individual silk proteins are known raw materials in cosmetic agents. Thus, sericin is offered as a commercial product, for example, by Pentapharm Ltd under the trade name Sericin Code 303-02. Fibroin as a protein hydrolyzate with different molecular weights is much more frequently available on the market. These hydrolyzates are commercialized in particular as “silk hydrolyzates”. Thus, hydrolyzed fibroin with average molecular weights from 350 to 1000 are commercialized, for example, under the trade name Promois® Silk.

Protein hydrolyzates of vegetal origin (e.g., soya-, almond-, pea-, potato- and wheat protein hyrolyzates) are available, for example, under the trade names Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

Although it is preferred to add the protein hydrolyzates as such, optionally other mixtures containing amino acids can also be added in their place. Likewise, it is possible to add derivatives of protein hydrolyzates, for example, in the form of their fatty acid condensation products. Such products are marketed, for example, under the trade names Lamepon® (Cognis), Lexein® (Inolex), Crolastin® (Croda), Crosilk® (Croda) or Crotein® (Croda).

Naturally, the teaching according to the invention includes all isomeric forms, such as cis/trans isomers, diastereoisomers and chiral isomers. According to the invention, it is also possible to employ a mixture of a plurality of protein hydrolyzates.

Agents according to the invention contain protein hydrolyzates, for example, in concentrations of 0.01 wt % to 20 wt %, preferably 0.05 wt % up to 15 wt % and quite particularly preferably in amounts of 0.05 wt % up to 5.0 wt %, based on total end-use preparation.

The agent can further contain at least one vitamin, one provitamin, one vitamin precursor and/or one of their derivatives as the care substance.

According to the invention, such vitamins, provitamins and vitamin precursors are preferred, which are normally classified in the groups A, B, C, E, F and H.

Retinol (vitamin A₁) as well as the 3,4-didehydroretinol (vitamin A₂) are classified as substances belonging to the vitamin A group. β-carotene is the provitamin of retinol. According to the invention, vitamin A acid and its esters, vitamin A aldehyde and vitamin A alcohol as well as its esters such as the palmitate and the acetate can be considered as the vitamin A component. The agents according to the invention preferably comprise the vitamin A components in amounts of 0.05 to 1 wt %, relative to the total ready for use preparation. The agents according to the invention preferably comprise vitamins, provitamins and vitamin precursors from groups A, B, C, E and H.

Panthenol, pantolactone, pyridoxine and its derivatives as well as nicotinamide and biotin are especially preferred.

D-panthenol is quite particularly preferably employed as a care substance, optionally in combination with at least one of the abovementioned silicone derivatives.

Like the addition of glycerin and/or propylene glycol, the addition of panthenol increases the flexibility of the polymer film that is formed when the agent according to the invention is used. Thus, if a particularly flexible hold is desired, then the agents can comprise panthenol instead of or in addition to glycerin and/or propylene glycol. In a preferred embodiment, the agents comprise panthenol, preferably in an amount of 0.05 to 10 wt %, particularly preferably 0.1 to 5 wt %, based on total agent.

Agents according to the invention can further comprise at least one plant extract as a care substance.

Usually, these extracts are manufactured by extraction of the whole plant. In individual cases, however, it can be preferred to produce the extracts solely from blossoms and/or leaves of the plant.

Regarding the inventively preferred plant extracts, reference is particularly made to extracts listed in the Table beginning on page 44 of the 3^{rd} Edition of the Guidelines for the Declaration of Ingredients in Cosmetics, (Leitfadens zur Inhaltsstoffdeklaration kosmetischer Mittel) published by the German Cosmetics, Toiletry, Perfumery and Detergent Association e.V. (IKW), Frankfurt.

According to the invention, mainly extracts from green tea, oak bark, stinging nettle, hamamelis, hops, henna, camomile, burdock root, field horsetail, hawthorn, linden flowers, almonds, aloe vera, spruce needles, horse chestnut, sandal wood, juniper, coconut, mango, apricot, lime, wheat, kiwi, melon, orange, grapefruit, sage, rosemary, birch, malva, lady's smock, common yarrow, thyme, lemon balm, rest-harrow, coltsfoot, marshmallow (althaea), meristem, ginseng and ginger are preferred.

Particularly preferred extracts are those from green tea, oak bark, stinging nettle, hamamelis, hops, field horsetail, hawthorn, linden flowers, almonds, aloe vera, coconut, mango, apricot, lime, wheat, kiwi, melon, orange, grapefruit, sage, rosemary, birch, malva, lady's smock, common yarrow, rest-harrow, meristem, ginseng and ginger.

Extracts of green tea, almonds, aloe vera, coconut, mango, apricot, lime, wheat, kiwi and melon are quite particularly suitable.

Extraction compositions used to prepare the cited plant extracts can be water, alcohols as well as their mixtures. Exemplary preferred alcohols are lower alcohols such as ethanol and isopropanol, particularly polyhydric alcohols such as ethylene glycol, propylene glycol and butylene glycol, both as the sole extraction agent as well as in aqueous mixtures. Plant extracts based on water/propylene glycol in the ratio 1:10 to 10:1 have proven to be particularly suitable. It is inventively possible in the context of the defined water quantity to add aqueous vegetal extracts. However, this is not inventively preferred.

According to the invention, the plant extracts can be used in pure as well as in diluted form. When they are used in diluted form, they normally comprise about 2-80% by weight active substance and the solvent is the extraction agent or mixture of extraction agents used for their extraction.

In addition, it can be preferred to employ mixtures of a plurality, particularly two different plant extracts in the agents according to the invention.

Compositions according to the invention preferably comprise these conditioners in amounts of 0.001 to 2, particularly 0.01 to 0.5 wt %, based on total preparation.

Mono or oligosaccharides can also be incorporated as the care substance into the agents according to the invention.

Both monosaccharides as well as oligosaccharides, such as raw sugar, lactose and raffinose, can be incorporated. According to the invention, use of monosaccharides is preferred. Once again, monosaccharides preferably include those compounds having or 6 carbon atoms.

Suitable pentoses and hexoses include ribose, arabinose, xylose, lyxose, allose, altrose, glucose, mannose, gulose, idose, galactose, talose, fucose and fructose. Arabinose, glucose, galactose and fructose are preferred incorporated carbohydrates; glucose is quite particularly preferably incorporated, and is suitable both in the D(+) or L(−) configuration or as the racemate.

In addition, derivatives of these pentoses and hexoses can also be incorporated according to the invention, such as the corresponding onic and uronic acids, sugar alcohols, and glycosides. Preferred sugar acids are the gluconic acid, the glucuronic acid, the sugar acids, the mannosugar acids and the mucic acids. Preferred sugar alcohols are sorbitol, mannitol and dulcitol. Preferred glycosides are the methyl glucosides.

As the added mono or oligosaccharides are usually extracted from natural raw materials such as starch, they generally exhibit the corresponding configurations (e.g., D-glucose, D-fructose and D-galactose).

The inventive agents preferably contain mono or oligosaccharides in an amount of 0.1 to 8 wt %, particularly preferably 1 to 5 wt %, based on total end-use preparation.

Although each of the cited care substances alone already provides a satisfactory result, in the context of the present invention all embodiments are also included, wherein the agent has a plurality of care substances even from different groups.

With addition of a UV filter, both the agent itself as well as the treated fibers can be protected against damage from UV radiation. Consequently, at least one UV filter is preferably added to the agent. Suitable UV filters are not generally limited in regard to their structure and their physical properties. Indeed, all UV filters that can be used in the cosmetic field having an absorption maximum in the UVA (315-400 nm), in the UVB (280-315 nm) or in the UVC (<280 nm) regions are suitable. UV filters having an absorption maximum in the UVB region, especially in the range from about 280 to about 300 nm, are particularly preferred.

Inventively preferred UV-filters are chosen from substituted benzophenones, p-aminobenzoates, diphenylacrylates, cinnamates, salicylates, benzimidazoles and o-aminobenzoates.

Those UV filters with a molecular extinction coefficient at the absorption maximum of above 15,000, particularly 20,000, are preferred.

Moreover, it was found that for structurally similar UV filters, in many cases in the context of the inventive teaching, the water-insoluble compound exhibits a higher activity than that of water-soluble compounds that differ from them by one or a plurality of additional ionic groups. In the context of the invention, water-insoluble UV filters are understood to mean those that dissolve not more than 1 wt %, especially not more than 0.1 wt % in water at 20° C. In addition, these compounds should be soluble to at least 0.1, especially to at least 1 wt % in conventional cosmetic oil components at room temperature. Accordingly, the use of water-insoluble UV filters can be inventively preferred.

The agent usually contains UV filters in amounts of 0.01 to 5 wt %, based on total end-use preparation. Quantities of 0.1-2.5 wt % are preferred.

In a particular embodiment, the agent further comprises one or more substantive dyes. Application of the agent then enables the treated keratinic fiber not only to be temporarily styled but also to be dyed at the same time. This can be particularly desirable when only a temporary dyeing is desired, for example, with flamboyant fashion colors that can be subsequently removed from the keratinic fibers by simply washing them out.

Substantive dyes are usually nitrophenylenediamines, nitroamino phenols, azo dyes, anthraquinones or indophenols.

Preferred substantive dyestuffs are compounds known under the international designations or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 11, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, and Acid Black 52 known compounds as well as 1,4-diamino-2-nitrobenzene, 2-amino-4-nitrophenol, 1,4-bis(β-hydroxyethyl)amino-2-nitrobenzene, 3-nitro-4(β-hydroxyethyl)aminophenol, 2-(2′-hydroxyethyl)amino-4-6-dinitrophenol, 1-(2′-hydroxyethyl)amino-4-methyl-2-nitrobenzene, 1-amino-4-(2′-hydroxyethyl)-amino-5-chloro-2-nitrobenzene, 4-amino-3-nitrophenol, 1-(2′-ureidoethyl)amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-1-hydroxy-4-nitrobenzene.

Cationic substantive dyes are preferably employed. Particular preference is given here to—
(a) cationic triphenylmethane dyes such as Basic Blue 7, Basic Blue 26, Basic Violet 2 and Basic Violet 14;
(b) aromatic systems which are substituted by a quaternary nitrogen group, such as Basic Yellow 57, Basic Red 76, Basic Blue 99, Basic Brown 16 and Basic Brown 17; and
(c) substantive dyes having a heterocycle that has at least one quaternary nitrogen atom, as described, for example, in EP 0 998 908 in claims 6 to 11.

Those dyes also known under the names Basic Yellow 87, Basic Orange 31 and Basic Red 51 are quite particularly preferred cationic substantive dyes of group (c).

Cationic substantive dyes that are commercialized under the trade name Arianor® are likewise quite particularly preferred cationic substantive dyes according to the invention.

Agents according to this embodiment contain substantive dyes preferably in an amount of 0.001 to 20 wt %, based on total agent.

In addition, compositions according to the invention can also comprise naturally occurring dyestuffs as found, for example, in henna red, henna neutral, henna black, camomile leaves, sandalwood, black tea, alder buckthorn bark, sage, logwood, madder root, cachou, cedar and alkanet root.

It is not required that each substantive dyestuff be pure compounds. In fact, compositions according to the invention, due to manufacturing processes for the individual dyestuffs, may contain minor quantities of additional components, so long as the latter have no detrimental influence on the styling result or must be excluded on other grounds (e.g., toxicological).

It is inventively preferred that the agents are exempt from oxidation dye precursors. Oxidation dye precursors are divided into developer components and coupler components. Under the influence of oxidizing agents or from atmospheric oxygen, the developer components form the actual colorants among each other or by coupling with one or more coupler components.

In addition to the cited components, the compositions can furthermore contain all active substances, additives and auxiliaries known for such preparations.

Further exemplary active products, auxiliaries and additives are—
thickeners like agar-agar, guar gum, alginates, xanthane gum, gum arabica, karaya gum, locust bean flour, linseed gums, dextrans, cellulose derivatives (e.g., methyl cellulose, hydroxyalkyl cellulose and carboxymethyl cellulose), starch fractions and derivatives such as amylose, amylopectin and dextrins, clays such as bentonite or synthetic hydrocolloids such as polyvinyl alcohol, and optionally crosslinked polyacrylates;
structurants such as maleic acid and lactic acid;
perfume oils, dimethyl isosorbitol and cyclodextrins;
defoamers such as silicones;
dyestuffs to color the composition;
anti-dandruff active materials such as Piroctone Olamine, zinc Omadine and Climbazole;
substances for adjusting the pH such as customary acids, particularly food acids, and bases;
texturizers such as sugar esters, polyol esters or polyol alkyl ethers;
complexants such as EDTA, NTA, β-alanine diacetic acid and phosphonic acids;
swelling and penetration substances such as glycerin, propylene glycol monoethyl ether, carbonates, hydrogen carbonates, guanidines, ureas, and primary, secondary and tertiary phosphates;
opacifiers such as latex, styrene/PVP copolymers and styrene/acrylamide copolymers;
pearlizing compositions such as ethylene glycol mono- and distearate as well as PEG-3 distearate;
preservatives;
antioxidants.

Regarding further optional ingredients and their amounts used, reference is expressly made to those relevant handbooks known to one skilled in the art.

Formulation of the inventive agents can be in all usual forms for styling agents, for example, gels, creams, solutions that can be applied as a lotion or pump spray or aerosol spray onto the hair, or other preparations suitable for application on the hair

The inventive agents are preferably made up as a pump spray, aerosol spray, pump foam or aerosol foam.

For such pump or aerosol forms, the agents according to the invention are packed in a dispensing device having either a pressurized gas container additionally containing a propellant (“aerosol container”) or by a non-aerosol container.

Pressurized gas containers wherein a product is dispersed through a valve by the internal gas pressure in the container are defined as “aerosol containers”. The opposite of the aerosol definition (i.e., a container under normal pressure) is defined as a “non-aerosol container”, from which a product is dispersed by the mechanical actuation of a pump system.

The agents are particularly preferably packed as an aerosol hair foam or aerosol hair spray. Consequently, the agent additionally comprises at least one propellant.

Inventive agents in the form of an aerosol product can be manufactured by known methods. Generally, all ingredients of the agent, excepting the propellant, are charged into a suitable pressure-resistant container. This is thereupon sealed with a valve. The desired quantity of propellant is then filled by means of conventional techniques.

In the embodiment, inventively suitable exemplary propellants are selected from N₂O, dimethyl ether, CO₂, air, alkanes containing 3 to 5 carbon atoms, such as propane, n-butane, iso-butane, n-pentane and iso-pentane, and their mixtures. Dimethyl ether, propane, n-butane, iso-butane and their mixtures are preferred. According to a preferred embodiment, the cited alkanes, mixtures of the cited alkanes or mixtures of the cited alkanes with dimethyl ether are preferred as the sole propellant. However, the invention also explicitly includes the joint utilization with propellants of the fluorochlorohydrocarbon type, but especially fluorinated hydrocarbons.

Inventive agents in the form of an aerosol spray preferably comprise the propellant in an amount of 30 to 60 wt %, based on weight of the whole agent.

Mixtures of propane and butane are quite particularly preferably used in the weight ratio propane to butane of 20 to 80 to 15 to 85 as the sole propellant. These mixtures are again preferably incorporated in compositions according to the invention in amounts of 30 to 55 wt %, based on weight of the total composition. According to the invention, butane refers to n-butane, iso-butane and mixtures of n-butane and iso-butane.

Inventively suitable exemplary propellants are chosen from N₂O, dimethyl ether, CO₂, air, alkanes containing 3 to 5 carbon atoms, such as propane, n-butane, iso-butane, n-pentane and iso-pentane, and their mixtures. Dimethyl ether, propane, n-butane, iso-butane and mixtures thereof are preferred.

The addition of the above cited additional ingredients and the added quantities or added quantity ratios classed above as preferred (vide supra) are of course also preferred in the context of this/these embodiment(s).

Inventively suitable exemplary propellants in the embodiment as aerosol foam are chosen from N₂O, dimethyl ether, CO₂, air, alkanes containing 3 to 5 carbon atoms, such as propane, n-butane, iso-butane, n-pentane and iso-pentane, and their mixtures. According to the embodiment of aerosol foam, the cited alkanes, mixtures of the cited alkanes or mixtures of the cited alkanes with dimethyl ether are employed as the sole propellant. However, the invention also explicitly includes the joint utilization with propellants of the fluorochlorohydrocarbon type, especially fluorinated hydrocarbons.

In regard to the weight ratio of propellant to the usual ingredients of the preparation, the size of the aerosol droplets or the foam bubbles and the relevant size distribution can be adjusted for a given spray device.

When a conventional aerosol device is used, aerosol foam products preferably comprise propellant in amounts of 1 to 35 wt %, based on total product. Quantities of 2 to 30 wt %, especially 3 to 15 wt %, are particularly preferred.

Agents in the form of gels are foamed in a two-chamber aerosol container, preferably with isopentane as the propellant, which is incorporated into the agent according to the invention and packed in the first chamber of the two-chamber aerosol container. At least one additional propellant that differs from isopentane is packed in the second chamber of the two-chamber aerosol container and generates a higher pressure than the isopentane. The propellants of the second chamber are preferably chosen from N₂O, dimethyl ether, CO₂, air, alkanes containing 3 or 4 carbon atoms (such as propane, n-butane, iso-butane) as well as mixtures thereof.

The addition of the previously cited additional preferred constituents and the added quantities or added quantity ratios characterized as preferred (see above) are of course preferred in the context of this embodiment.

Agents according to the invention and products containing these agents, especially aerosol hair foams and aerosol hair sprays, lend a strong hold and volume to the treated hair.

A second subject matter of the invention is a cosmetic composition containing, in a cosmetic carrier—
(a) at least one polyamide that is a reaction product of at least one dimerized fatty acid and at least one diamino compound, and
(b) at least one propellant gas.

Here, those embodiments in regard to the stated polyamide or to the preferred additional additives cited in the first subject matter of the invention are likewise preferably suitable for use in cosmetic agents with at least one propellant gas.

Preferred cosmetic agents of this embodiment are those in the form of an aerosol spray or aerosol foam.

Those embodiments according to the invention comprising these cosmetic agents, especially aerosol hair foams or aerosol hair sprays, lend to the treated hair a very strong, long-lasting hold to the hairstyle while the hair remains flexible.

A third subject matter of the invention is a method for shaping hair, wherein a cosmetic agent comprising in a cosmetic carrier at least one polyamide that is a reaction product of at least one dimerized fatty acid and at least one diamino compound is applied onto the hair and the hair, before or during the application, is set into a hairstyle.

In this regard, the embodiments in regard to the cosmetic agent cited in the first subject matter of the invention are likewise preferred.

In this regard it is inventively preferred to shape the hair and to fix this shape by the stated cosmetic agent.

Furthermore, it is preferred, once the stated cosmetic has been applied on the hair, to leave it there (i.e., not to rinse it out again).

The abovementioned dispensing devices or aerosol products (see above) are inventively preferred application aids.

The following examples are intended to illustrate the subject matter of the present invention in more detail, without limiting it in any way.

### EXAMPLES

Unless otherwise stated, the quantities are understood to be in weight percent of the active substance.

The following compositions were prepared:

| Raw material | E1 | E2 | E3 | E4 | E5 | V1 |
|---|---|---|---|---|---|---|
| PA1 | 5.0 | — | — | — | — | — |
| PA2 | — | 5.0 | — | — | — | — |
| PA3 | — | — | 5.0 | — | — | — |
| PA4 | — | — | — | 5.0 | — | — |
| PA5 | — | — | — | — | 5.0 | — |
| Amphomer ® | — | — | — | — | — | 5.0 |
| Isopropanol/Hexane* | 95.0 | 95.0 | 95.0 | 95.0 | 95.0 | 95.0 |

*in the weight ratio 2 to 1
PA1: polyamide obtained by polymerizing a dimerized fatty acid (having 36 carbon atoms) with 1,2-ethylenediamine, 1,10-diaminodecane and a diaminopolyether (acid number: 1.4; amine number: 6.8; glass transition temperature: −15° C., elastic modulus: 30; yield MPa: 4.5: break MPa: 10, elongation %: 600)
PA2: polyamide obtained by polymerizing a dimerized fatty acid (having 36 carbon atoms) with 1,2-ethylenediamine, 1,6-diaminohexane (acid number: 0.8; amine number: 5.5; glass transition temperature: −10° C., elastic modulus: 130; yield MPa: 8.0: break MPa: 10, elongation %: 500)
PA3: polyamide obtained by polymerizing a dimerized fatty acid (having 36 carbon atoms) with 1,6-diaminohexane (acid number: 0.05; amine number: 2.6; elastic modulus: 100; yield MPa: 9.9: break MPa: 26, elongation %: 580)
PA4: polyamide obtained by polymerizing a dimerized fatty acid (having 36 carbon atoms) with 1,2-ethylenediamine (acid number: 4.3; amine number: 1.6; glass transition temperature: 5° C., elastic modulus: 320; yield MPa: 13; break MPa: 12, elongation %: 20)
PA5: polyamide obtained by polymerizing a dimerized fatty acid (having 36 carbon atoms) with 1,2-ethylenediamine, 1,9-diaminononane and a diaminopolyether (acid number: 6.5; amine number: 0.7; glass transition temperature: −35° C., elastic modulus: 85; yield MPa: 6: break MPa: 9, elongation %: 600)

### 1.0 Determination of the High Humidity Curl Retention (HHCR)—

Standardized strands of hair from Kerling Co. (art. no. 827560) of the hair type “European Natural”, color 6/0) length (Lₘₐₓ) 220 mm and weight 0.6 g were used.

The strands were prepared by washing them with a 12.5% conc. sodium laureth sulfate solution. The strands of hair were dried overnight in a drying oven at 318° K.

The compositions (0.18 g) were each applied onto a strand of hair and massaged in. The strands were then wrapped onto a winder (Fripac-medis, Ø7 mm, art. no. D-1203) and dried overnight at room temperature.

The winders were then carefully removed and the strands were suspended. The lengths of the strands were each measured (L₀) and the strands were placed into a climate chamber. They were stored there at 294 K and a relative air humidity of 85% for a period of 6 hours, after which the lengths of the strands were remeasured (Lₜ).

Five (5) test strands per composition were treated in the same way and measured.

High Humidity Curl Retention (HHCR) was calculated using the following formula and expressed as the arithmetic mean of the 5 samples of the HHCR values for each composition:

$$HHCR = \frac{{L}_{max} - {L}_{t}}{{L}_{max} - {L}_{0}}$$

### 2.0 Determination of Elasticity—

A dry tress of hair (Euro-Naturhaar from the Kerling Company, adhesive tress compacted, adhesive on one side, total length 150 mm, free length 130 mm, width 10 mm, weight 0.9±0.1 g) was dipped for 30 seconds up to the lower edge of the mask into the test polymer solution. The excess solution was then wiped off between thumb and index finger, such that there remained 0.5±0.02 g of the solution on the hair. The tress of hair that was saturated with the test solution was wrapped round a Teflon cylinder with a diameter of 36 mm, and the projecting ends were fixed with a clip. The prepared strands were then dried and conditioned overnight (14 hours) in the climatic test cabinet at 25° C. and 50% relative humidity or at 25° C. and 75% relative humidity.

The conditioned strand was carefully removed from the Teflon cylinder. The resulting Ω-Loop, a circular structure of the hair, stabilized in its shape by the formed polymer film, was clamped in the gripper attached to the load cell and lowered close above the base plate of a universal testing machine AMETEK LF Plus from AMETEK Precision Instruments Europe GmbH, Product group Lloyd. The complete measurement was carried out in the climatic test cabinet under constant climatic conditions at 25° C. and 50% relative humidity.

In order to create standardized starting conditions, the measurement began with the start-up of an initial load of 0.07 N with a speed of 30 mm min⁻¹. The Ω-Loop was then compressed by 8 mm with a speed of 60 mm min⁻¹, the required force for this being measured. Once the characteristic force F₁ at the maximum deformation of 8 mm had been recorded, the strain on the strand was relieved at 60 mm min⁻¹ so far that the strand lifted 10 mm from the base plate. From this point on begins the next cycle, in that the initial load of 0.07 N is again initialized and the strand is then compressed by 8 mm, the same speeds being used as described above. The measurement of an Ω-Loop includes a total of 10 cycles.

Elasticity (Elastizität) was calculated from the measured forces according to the following formula:

$$Elastizit \ddot{a} t = \frac{\frac{{F}_{10} ( 2 mm ) - {F}_{10} ( 1 , 5 mm )}{0 , 5}}{\frac{{F}_{1} ( 2 mm ) - {F}_{1} ( 1 , 5 mm )}{0 , 5}} = \frac{{E}_{10}}{{E}_{1}}$$

To calculate elasticity, the deformation forces are determined for deformations of 1.5 mm and 2 mm, respectively, from the first and tenth cycles and ratioed.

TABLE 1
Results

| Composition | HHCR 6 h (%) | Elasticity (%) |
|---|---|---|
| E1 | 90 | 65 |
| E2 | 95 | 60 |
| E3 | 95 | 70 |
| E4 | 99 | 60 |
| E5 | 86 | 70 |
| V1 | 86 | 40 |

In the context of the shape fixing of a hairstyle, the inventive agents E1 to E4 achieved a better hairstyle hold and better elasticity of the hairstyle than a non-inventive styling composition VI containing the commercial polymer Amphomer.

We claim:

1. Method of fixing the shape of a hairstyle comprising:
applying to hair a cosmetic agent comprising, in a cosmetic carrier:
at least one polyamide that is a reaction product of at least one dimerized fatty acid and at least one diamino compound.

2. Method of fixing the shape of a hairstyle according to claim 1 further comprising preparing the dimerized fatty acid by coupling unsaturated (C₁₀ to C₂₄) monocarboxylic acids

3. Method of fixing the shape of a hairstyle according to claim 2, wherein the unsaturated (C₁₀ to C₂₄) monocarboxylic acids are linoleic acid, linolenic acid and/or oleic acid.

4. Method of fixing the shape of a hairstyle according to claim 1, wherein the diamino compound is chosen from at least one compound of Formula (I)
H₂N—R¹—NH₂ (I)
wherein R¹ is a linear (C₂ to C₁₀) alkylene group, a branched (C₂ to C₁₀) alkylene group, a *—R²—O—(CH₂CH₂O)ₙ(CH₂CHMeO)ₘ—R³* group wherein R² and R³ are, independently of one another, a (C₂ to C₁₀) alkylene group, and n and m are, independently of one another, an integer from 0 to 100, wherein the sum of m+n>0, or a group of Formula 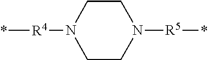
wherein R⁴ and R⁵ are, independently of one another, a (C₂ to C₆) alkylene group.

5. Method of fixing the shape of a hairstyle according to claim 4, wherein R² and R³ are ethane-1,2-diyl or propane-1,2-diyl).

6. Method of fixing the shape of a hairstyle according to claim 1, wherein the diamino compound is a combination of at least one compound of Formula (I) and at least one compound of Formula (I-1)
H₂N—R¹—NH₂ (I)
H₂N—R²—O—(CH₂CH₂O)ₙ(CH₂CHMeO)ₘ—R³—NH₂ (I-1)
wherein R¹ is a (C₂ to C₁₀) alkylene group, R² and R³ are, independently of one another, a (C₂ to C₁₀) alkylene group, and n and m are, independently of one another, an integer from 0 to 100, wherein the sum of m+n>0.

7. Method of fixing the shape of a hairstyle according to claim 1, wherein the at least one diamino compound is at least 1,2-ethylenediamine.

8. Method of fixing the shape of a hairstyle according to claim 1, wherein the polyamide has an E-modulus at 2% deformation from 10 to 500.

9. Method of fixing the shape of a hairstyle according to claim 1, wherein the polyamide has an elongation at break in % from 20 to 1000.

10. Method of fixing the shape of a hairstyle according to claim 1, wherein the polyamide has an acid number of 0.01 to 8.

11. Cosmetic composition comprising, in a cosmetic carrier:
(a) at least one polyamide that is a reaction product of at least one dimerized fatty acid and at least one diamino compound, and
(b) at least one propellant gas.

12. Process for shaping hair comprising:
applying onto the hair a cosmetic agent comprising, in a cosmetic carrier, at least one polyamide that is a reaction product of at least one dimerized fatty acid and at least one diamino compound, and
setting the hair into a hairstyle before or during the application.
